(19) United States
(12) Patent Application Publication
(10) Pub. No.: US 2025/0222205 A1
(43) Pub. Date: Jul. 10, 2025

(54) METHOD AND APPARATUS FOR BUFFERING ANESTHETICS

(71) Applicant: ONPHARMA COMPANY, Carson City, NV (US)

(72) Inventors: Jeffrey J. Christian, Morgan Hill, CA (US); Zach Taylor, Pinehurst, NC (US); Michael Falkel, Carmel, CA (US); Bryant Grigsby, Aptos, CA (US); Scott Arnett, Springville, UT (US); Connor Wilson, Los Alamos, NM (US)

(21) Appl. No.: 19/092,036

(22) Filed: Mar. 27, 2025

Related U.S. Application Data
(63) Continuation of application No. PCT/US2023/075114, filed on Sep. 26, 2023.
(60) Provisional application No. 63/410,524, filed on Sep. 27, 2022.

(51) Int. Cl. A61M 5/24; A61M 5/19; A61M 5/315; A61M 19/00

(52) U.S. Cl. CPC A61M 5/2448; A61M 5/19; A61M 5/2422; A61M 5/31586; A61M 19/00; A61M 2005/2474; A61M 2205/581

(57) ABSTRACT

A device is disclosed for compounding two solutions contained in cartridges, where one of the cartridges serves as the mixing vessel. The device can be used for buffering anesthetic prior to use by a medical practitioner, including, but not limited to, dentists buffering local anesthetic prior to use. In various embodiments, a container of buffering solution can be incorporated into the device, which will maintain the buffer in a stable condition. The device can be used to buffer anesthetic in one or more cartridges and when the buffer in the device is exhausted, the device can be reloaded or discarded.

### CROSS-REFERENCE TO RELATED APPLICATIONS

This patent application is a continuation of International Application No. PCT/US2023/075114, filed Sep. 26, 2023, which claims the benefit of U.S. Patent Application No. 63/410,524 filed Sep. 27, 2022, each of which are incorporated herein by reference in its entirety.

### FIELD OF THE INVENTION

The field of the invention is medical devices. Specifically, the field is medical devices for buffering anesthetic.

### BACKGROUND OF THE INVENTION

Local anesthetics, such as lidocaine, typically in combination with epinephrine in an aqueous solution, are commonly used in dentistry to numb a patient's tissues prior to performing various procedures, such as filling teeth, emplacement of crowns, or endodontic procedures, among others.

Without buffering, aqueous solutions containing lidocaine and epinephrine typically have a pH about 3.5. At that pH, the anesthetic typically burns and stings the patient. At pH 3.5, the amount of lipid soluble anesthetic molecules (the base or “active” form) capable of passing through the nerve membrane is about 1 in 25,000 molecules, which is too low to lead to anesthesia in the nerve. At neutral pH, the proportion of the active form is 1 in 4, or about 6000 times greater, which is sufficient to lead to anesthesia in view of considerations described in the following paragraphs.

It can take as long as 45 minutes for the patient's buffering capacity to raise the pH of injected anesthetic solutions to a point where there is sufficient active anesthetic available to cross the nerve membrane and create analgesia. Further, some patients in some cases do not have the buffering capacity to raise the pH to a point where analgesia is achieved.

Buffering the anesthetic to neutral pH with bicarbonate solutions before injection not only immediately increases the concentration of active anesthetic molecules in the solution, but also provides additional anesthetic properties from dissolved CO₂. Also, after entering the nerve, the base form of the anesthetic must be converted back to its acid form to block nerve receptor sites. High levels of CO₂ inside the nerve create an acidic environment, thus converting the base form of the anesthetic to its acidic form after the base form has successfully crossed the nerve membrane. As receptor sites are blocked in the nerve, the nerve's ability to transmit pain signals is reduced.

Aqueous solutions containing bicarbonate ions are used in various medical applications including, but not limited to, antidotes, dialysates, artificial cerebrospinal fluid, intraocular irrigating solutions, cardiac perfusates, cardioplegic solutions, peritoneal irrigating solutions, and solutions for organ preservation. Of particular interest to the present application, bicarbonate solutions are used to buffer dental and other anesthetics to control pH. One of the most commonly used medical bicarbonate solutions consists of sodium bicarbonate (NaHCO₃) mixed with water (H₂O). In medical bicarbonate solutions, bicarbonate ions are in equilibrium as represented by the following expression:

2HCO₃⁻↔CO₂ ↑+CO₃²⁻+H₂O

If the reaction occurs in a closed system, equilibrium is reached with the amounts of reactants remaining constant. In open systems, however, the carbon dioxide gas escapes and the reaction proceeds from the left to the right with bicarbonate (2HCO₃) evolving into carbon dioxide gas (CO₂), carbonate (CO₃) and water (H₂O), progressively decreasing the concentration of bicarbonate ions and increasing the concentration of carbonate ions. Since carbonate ions are more alkaline than bicarbonate ions, the pH of the solution will progressively increase as CO₂ evolves out of solution.

Clinical effectiveness of bicarbonate medical solutions often depends on maintenance of a particular pH range, generally from 7 to 9. For some applications, maintaining the pH in a narrower range is beneficial. To stabilize pH and CO₂ content, sodium bicarbonate solutions are conventionally packed in gas tight containers that limit the evolution of CO₂ out of solution and into the atmosphere. By limiting the loss of evolved CO₂, pH change can be reduced. As CO₂ leaves solution and enters the container's “headspace” (the gas-filled region above the solution), the partial pressure of the evolved CO₂ will increase and eventually establish equilibrium between CO₂ leaving solution and CO₂ returning to solution.

The gas tight container most commonly used to store medical bicarbonate solutions is the glass vial with a pierceable rubber cap, the cap being referred to as a septum. Such vials allow the medical practitioner to pierce the septum with a hypodermic needle and withdraw or “draw up” a desired volume of bicarbonate solution into a syringe. To facilitate withdrawing the bicarbonate, the vials typically include a significant headspace that prevents a vacuum lock from forming when the practitioner attempts to draw up the fluid, as the gas in the headspace can expand to compensate for the fluid being removed. Once the bicarbonate solution is drawn up into a syringe, the syringe can be used to deliver the fluid into another container, a catheter or a blood vessel. A partially filled syringe can be used to draw up a second solution, including but not limited to, a local anesthetic, from another vial in order to mix the second solution with the sodium bicarbonate, where the syringe serves as a mixing and delivery vessel for the resulting pH buffered local anesthetic solution. One drawback to using such vial-and-syringe systems for storing, mixing, and/or delivering bicarbonate solutions is that drawing up the solution from a vial into the syringe reduces the partial pressure of the CO₂ in the syringe which causes CO₂ to leave solution and create CO₂ bubbles in the solution during the transfer. Also, there can be significant agitation of the solution as the bubbles enter the syringe, further causing CO₂ to dissolve out of solution. Likewise, drawing up solution from a vial causes a drop in the partial pressure of the CO₂ in the vial's headspace, which results in CO₂ leaving solution and entering the headspace to re-establish equilibrium between the partial pressure of CO₂.in solution and in the headspace. In this manner, with each dose of bicarbonate removed from the vial, CO₂ leaves solution and enters the headspace, causing the pH of the bicarbonate solution remaining in the vial to increase. For these reasons, even if the pH of a sodium bicarbonate buffering solution in a vial-type storage container were estimated or ascertained before delivery, drawing up, mixing and/or delivery of the bicarbonate system can alter the pH of the solution to an undesirable extent.

One particular device for combining a buffer solution, including but not limited to, sodium bicarbonate, with an anesthetic, including, but not limited to, a dental anesthetic in a conventional cartridge, is described in U.S. Pat. No. 5,603,695. The device comprises a buffer cartridge having a needle which can be penetrated through the septum of the anesthetic cartridge. The buffer is stored in a cartridge with significant head space and no provision for maintaining volatile CO₂ in solution in a bicarbonate anesthetic. Moreover, no provision is made for displacing anesthetic from the anesthetic cartridge as the buffer is introduced.

For these reasons, it would be desirable to provide improved methods and apparatus for combining buffer solutions with anesthetics or other medical solutions, particularly where the buffer solutions are held in conventional glass cartridges. It would be particularly beneficial if the methods and devices employed buffer cartridges which maintained the buffer solution, more particularly sodium bicarbonate solution, in a stable condition with minimal pH change and carbon dioxide loss prior to use.

### SUMMARY OF THE INVENTION

Some embodiments of the present disclosure relate to a device. The device can comprise a buffer compartment configured to receive a buffer cartridge comprising a first septum and a plunger, and containing a buffer.

The device can comprise an anesthetic compartment configured to receive an anesthetic cartridge comprising a second septum and containing an anesthetic.

The device can also comprise an overflow compartment. In some embodiments, an overflow compartment can comprise an absorbent member configured to absorb at least some fluid expelled from the anesthetic cartridge. Additionally or alternatively, the overflow compartment can comprise a plurality of granules configured to absorb fluid, including, but not limited to, fluid not absorbed or not retained by the absorbent member, if the absorbent member is present.

In some embodiments, the buffer compartment can be proximal a first end of the device, the anesthetic compartment can be proximal a second end of the device, and the overflow compartment can be positioned between the buffer compartment and the anesthetic compartment.

The device can also comprise a needle assembly, comprising a transfer needle having a first end and a second end and an exhaust needle having a first end and a second end; a compressible member configured to compress upon reception of the anesthetic cartridge by the anesthetic compartment, wherein compression of the compressible member causes the first end of the transfer needle to penetrate through the first septum, the second end of the transfer needle to penetrate through the second septum, the second end of the exhaust needle to penetrate through the second septum, and wherein the first end of the exhaust needle is present in the overflow compartment. In some embodiments, the first end of the exhaust needle enters the overflow compartment. In some embodiments, the first end of the exhaust needle is already present in the overflow compartment. In some embodiments, the second end of the transfer need extends further into the anesthetic cartridge than the second end of the exhaust needle. In some embodiments, an anesthetic compartment can comprise at least one retaining member configured to retain an anesthetic cartridge after reception such that a compressible member remains compressed until the anesthetic cartridge is removed from the anesthetic compartment. In some embodiments, when the anesthetic cartridge is removed from the anesthetic compartment, and the compressible member is no longer compressed by the anesthetic cartridge, the first end of the transfer needle is withdrawn from the first septum, the second end of the transfer needle is withdrawn from the second septum, the second end of the exhaust needle is withdrawn from the second septum. In some embodiments, when the anesthetic cartridge is removed from the anesthetic compartment, the first end of the exhaust needle is also withdrawn from the overflow compartment. In an embodiment, the first end of the exhaust needle stays in the overflow compartment. In an embodiment, the first end of the exhaust needle is present in the overflow compartment at all times. In an embodiment, the transfer needle is offset to ensure effective compounding. In an embodiment, the second end of the transfer needle extends further into the interior volume of the anesthetic cartridge than the second end of exhaust needle.

The device can also comprise an advancer member configured to engage with the plunger in the buffer cartridge such that upon reception of the anesthetic cartridge by the anesthetic compartment, an advancement of the advancer member expels buffer from the buffer cartridge into the anesthetic cartridge via the transfer needle, and whereby fluid is expelled from the anesthetic cartridge into the overflow compartment via the exhaust needle. For example, the advancer member can comprise a dial, wherein rotation of the dial advances the plunger. Alternatively or in addition, the advancer member can comprise a ratchet and pawl mechanism configured to ensure that the dial can only turn in one direction, and expels a predetermined dose of the buffer upon a rotation or a partial rotation of the dial. Further alternatively or additionally, the ratchet and pawl mechanism can be configured such that the ratchet and pawl mechanism is only engaged and can only function to expel a dose of buffer when an anesthetic cartridge is inserted in the anesthetic compartment. Further alternatively or additionally, the advancer member can comprise a plurality of detent notches, e.g., dentate notches. Further or additionally, the device can include a clutch, which would slip in the event that the advancer member is engaged when there is no anesthetic cartridge in the anesthetic compartment or the transfer needle is not engaged with the buffer cartridge. This can reduce or prevent an overpressure that can break one or more components of the device. Further, the clutch, or slip clutch, would allow frictional forces to lock the distal portion of the screw with the external knob to allow buffer solution to be expended from the device. Further, the detent notches would be configured such that the dial can only be rotated in one direction by the user. The device can include an arrow or other symbol indicating to the user the direction that the external knob should be rotated in order to operate the device.

Further, or in addition, the ratchet or clutch mechanism can be configured to provide a tactile or an audible indicator when the dial has been rotated from its start position to its end position for one operation of the device.

In some embodiments, the device can be used to operate with multiple instances of buffering anesthetic cartridges, with each instance further depleting the buffer cartridge. In some or additional embodiments, the device can contain an indicator of the remaining number of available instances before the buffer cartridge has been exhausted. In one embodiment, the device contains a window through which the user can view the advancer member and determine by an indicator on the device whether additional uses remain or if the device has delivered its last use. In some embodiments, the device can be cylindrical.

Some embodiments of the present disclosure relate to a method. The method can comprise providing a device: providing a buffer cartridge comprising a first septum, a plunger, and containing a buffer; providing an anesthetic cartridge comprising a second septum and containing an anesthetic; providing a needle assembly, comprising a transfer needle having a first end and a second end and an exhaust needle having a first end and a second end; and attaching the anesthetic cartridge to the device; compressing a compressible member and penetrating the first end of the transfer needle through the first septum, the second end of the transfer needle through the second septum, the second end of the exhaust needle through the second septum, and wherein the first end of the exhaust needle is present in the overflow compartment. In some embodiments, the first end of the exhaust needle enters the overflow compartment. In some embodiments, the first end of the exhaust needle is already present in the overflow compartment.

The method can comprise advancing the plunger, thereby expelling buffer from the buffer cartridge into the anesthetic cartridge via the transfer needle, whereby fluid is expelled from the anesthetic cartridge into the overflow compartment via the exhaust needle. In some embodiments, the transfer needle extends further into the anesthetic cartridge than the exhaust needle such that when the buffer is expelled into the anesthetic cartridge, the fluid that is simultaneously expelled through the exhaust needle is mostly comprised of local anesthetic.

In some embodiments, advancing the plunger can comprise rotating a dial. In some further embodiments, rotating the dial can engage a ratchet and pawl mechanism, and expelling the buffer can comprise expelling a predetermined dose of the buffer upon engagement of the ratchet and pawl mechanism. In a further embodiment, the mechanism placed against the plunger during assembly of the device prevents the plunger in the buffer cartridge from moving outward, thereby preventing gas from leaving solution in the buffer cartridge and forming a headspace in the buffer cartridge. In another embodiment, the mechanism placed against the plunger creates pressure in the buffer cartridge, also preventing gas from leaving solution and forming a headspace in the buffer cartridge. In a further embodiment, a slip clutch mechanism can be provided to hold the plunger in place and/or facilitate pressurization of the buffer during assembly of the device.

The method can further comprise absorbing at least some fluid expelled into the overflow compartment by an absorber member, and/or absorbing fluid not absorbed or not retained by the absorbent member, by a plurality of granules.

Alternatively or in addition, the method can further comprise retaining the anesthetic cartridge in a position to maintain the compressing of the compressible member.

In some embodiments, the method can further comprise withdrawing the anesthetic cartridge from the device. In an embodiment, the method can further comprise removal of the transfer needle from the buffer cartridge when the anesthetic cartridge is disconnected from the device. In an embodiment, the transfer needle is offset to ensure effective compounding. In still further embodiments, the method can further comprise providing a second or additional instances of the anesthetic cartridge; and repeating the attaching, compressing, advancing, transfer and removal described above with the second or additional instances of the anesthetic cartridge. For example, the buffer cartridge, the needle assembly, the compressible member, and the advancer member configured for advancing the plunger, can be positioned within a housing, and the method can further comprise discarding the device when the buffer cartridge is substantially empty of buffer. In an embodiment, the anesthetic cartridge locks in place when inserted correctly. In an embodiment, correct insertion is accompanied by an audible click. In an embodiment, the anesthetic cartridge is inserted by placing the anesthetic cartridge into the device and applying gentle pressure until the anesthetic cartridge locks in place.

Some embodiments of the present disclosure relate to a second method. The second method can comprise providing a device, including, but not limited to, the device summarized above; and inserting the buffer cartridge into the buffer compartment. The second method can further comprise, after inserting the buffer cartridge, packaging the device in combination with instructions for performing the method summarized above. In some embodiments, the instructions can further comprise instructions for inserting and withdrawing the anesthetic cartridge from the device. In some still further embodiments, the instructions can further comprise instructions for providing a second or additional instances of the anesthetic cartridge; and repeating the compressing and advancing with the second or additional instances of the anesthetic cartridge.

The devices and methods disclosed herein can provide buffer solutions, including, but not limited to, sodium bicarbonate solutions, in a stable condition with minimal pH change and carbon dioxide loss prior to use. They can also provide for introducing and combining the buffer solutions with anesthetic solution, where the anesthetic solution is held in conventional cartridges, without delivering an excess volume of buffer to the anesthetic cartridge, and relieving or exhausting an equal volume of anesthetic from the cartridge. Also, the devices can be provided to a practitioner, including, but not limited to, a dentist, in a ready-to-use form. Also, the devices and methods can provide for combining the buffer solutions with local anesthetics that have been formulated as hypoosmotic solutions, such that the addition of hyperosmotic buffer solutions will bring the buffered anesthetic toward iso-osmolarity, or towards a osmolarity closer to that of the human body. In an embodiment, the buffer is hyperosmotic and the anesthetic is hypoosmotic, such that when the buffer and anesthetic are combined the combination has an intended target osmolarity. In an embodiment, the target osmolarity is between 200 mOsm/L and 416 mOsm/L. In an embodiment, the target osmolarity is substantially isosmotic at 308 mOsm/L.

In an embodiment, the method comprises compounding a medication.

### Incorporation by Reference

All publications, patents, and patent applications mentioned in this specification, including, but not limited to, U.S. Pat. Nos. 8,162,917; 8,690,853; and 8,303,566, are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts an external perspective view of a device.

FIG. 2 depicts a cut-away perspective view of the device.

FIG. 3 depicts a cross-sectional view of a portion of the device. The cross-sectional view is “nearsighted” and “farsighted” in that it depicts portions of some elements that are behind or in front of the cross-section and would not be visible in a strict cross-sectional view.

FIG. 4A depicts a cut-away perspective view of a portion of the device during a first stage of use before an anesthetic cartridge is connected.

FIG. 4B depicts a cut-away perspective view of a portion of the device in a second stage of use when an anesthetic cartridge has been connected and is fully seated in the anesthetic compartment.

FIG. 4C depicts a cut-away perspective view of a portion of the device, including retaining members.

FIG. 5A depicts a perspective view of a portion of the device at a first stage of a second use.

FIG. 5B depicts a perspective view of a portion of the device at a second stage of a second use.

FIG. 6 depicts a cut-away perspective view of a portion of the device.

FIG. 7 depicts a cut-away perspective view of a portion of the device.

FIG. 8 depicts a longitudinal cross-sectional view of the distal portion of the device.

FIG. 9 depicts a cross-sectional view perpendicular to the longitudinal cross-section depicted in FIG. 8.

### DETAILED DESCRIPTION OF THE INVENTION

Unless defined otherwise, all technical and scientific terms used herein have the same meaning as is commonly understood by one of skill in the art to which this disclosure belongs.

The present invention provides methods and apparatus for buffering anesthetics or other medical solutions. The present device is useful for, among other things, compounding anesthetics and other therapeutics. Previously, anesthetics or other medical solutions could be buffered by being held in a conventional cartridge, particularly those having a penetrable septum and a slidable plunger or plug, including, but not limited to those cartridges generally described in U.S. Pat. No. 5,603,695, the full disclosure of which is incorporated herein by reference. Such cartridges are commonly used in dental practice, particularly disposable cartridges containing local anesthetics that are loaded into a reusable syringe to which a disposable needle had been attached, and the assembly is then used to deliver local anesthetic to a patient's tissue to create analgesia prior to a dental procedure. Such disposable anesthetic cartridges are conventionally loaded into a reusable stainless steel dental syringe or other delivery device, where the syringe engages the plunger in the cartridge to dispense the anesthetic through a disposable needle attached the syringe, one end of such needle having penetrated the anesthetic cartridge through its septum when the cartridge was loaded into the dental syringe. In this way a cartridge can be loaded into the syringe, which is then used to deliver the local anesthetic via an injection. The cartridge and disposable needle can be removed from the syringe and discarded, the syringe remaining available to be reloaded and used again for that procedure or sterilized and used for a later procedure.

To optimize effectiveness of the anesthetics and to reduce injection pain, it is desirable to buffer conventional anesthetics toward physiologic pH shortly before use. Typically dental anesthetics including, but not limited to, lidocaine, articaine, prilocaine, or mepivacaine, are formulated and stored in an acid form to promote shelf life. In order to consistently and reliably use sodium bicarbonate solution, or other buffering solutions, it is very important, that the buffering solutions themselves have predictable, stable pHs and chemical compositions in order for the buffered anesthetic to consistently, and in a controlled manner, buffer the local anesthetic toward physiologic pH and reliably achieve an optimum effect and minimum injection pain.

A container that holds the solution under pressure or that does not allow a headspace to form easily will prevent pH change from occurring by preventing CO₂ from leaving solution. In an application where it is desirable to have minimal or zero change in the pH of the solution being used as a buffer, preventing CO₂ from leaving solution each time the solution is sourced as a buffer would be desirable.

It would be desirable to provide improved methods and apparatus for combining buffer solutions with anesthetics or other medical solutions, particularly where the buffer solutions are held in conventional glass cartridges. It would be particularly beneficial if the methods and devices employed buffer cartridges which maintained the buffer solution, more particularly sodium bicarbonate solution, in a stable condition with minimal pH change and carbon dioxide loss prior to use. It would be still further desirable if the methods and systems provided for introducing and combining the buffer solutions with anesthetic solution, where the anesthetic solution is held in conventional cartridges, without delivering an excess volume of buffer to the anesthetic cartridge, and relieving or exhausting an equal volume of anesthetic from the cartridge. It would be further desirable if the systems could be provided to a practitioner, including, but not limited to, a dentist, in a ready-to-use form.

In embodiments, the present disclosure relates to a device, comprising a buffer compartment configured to receive a buffer cartridge comprising a first septum and a plunger, and containing a buffer; an anesthetic compartment configured to receive an anesthetic cartridge comprising a second septum and containing an anesthetic; an overflow compartment; a needle assembly, comprising a transfer needle having a first end and a second end and an exhaust needle having a first end and a second end; a compressible member configured to compress upon reception of the anesthetic cartridge by the anesthetic compartment, wherein compression of the compressible member penetrates the first end of the transfer needle through the first septum, the second end of the transfer needle through the second septum, the second end of the exhaust needle through the second septum, and wherein the first end of the exhaust needle is present in the overflow compartment, and an advancer member configured to engage with the plunger upon reception of the anesthetic cartridge by the anesthetic compartment, wherein advancement of the advancer member expels buffer from the buffer cartridge into the anesthetic cartridge via the transfer needle, whereby fluid is expelled from the anesthetic cartridge into the overflow compartment via the exhaust needle. In some embodiments, the first end of the exhaust needle enters the overflow compartment. In some embodiments, the first end of the exhaust needle is already present in the overflow compartment.

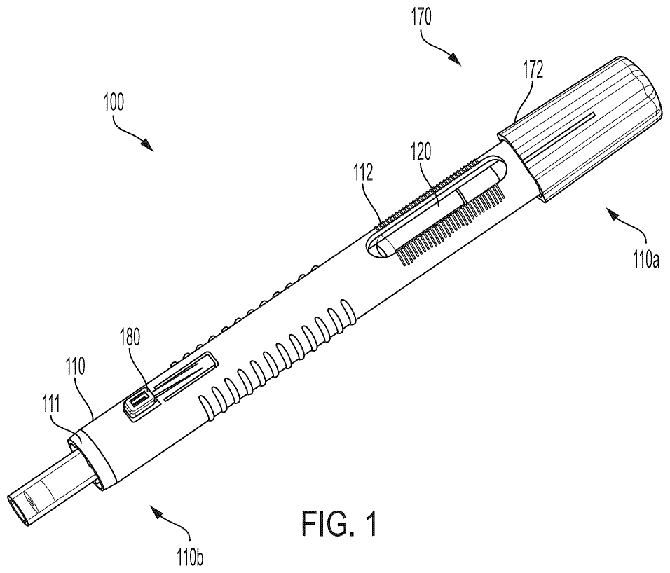

FIG. 1 shows a perspective view of an exemplary device 100. The device 100 comprises a housing 110. The housing has a first end 110a and a second end 110b. The housing 110 can be made of plastic, metal, other materials, or any combination thereof. Desirably, the housing 110 can be made from material(s) that can be readily sterilized.

In embodiments wherein the second end 110b is open, including, but not limited to, that shown in FIG. 1, the housing 110 can comprise a reinforcement band 111 positioned around the opening of the second end 110b. The reinforcement band 111 can comprise a metal.

In the embodiment shown in FIG. 1, the housing 110 comprises a window 112. In an embodiment, the window 112 can be an absence of material. In other embodiments, the window 112 can be made from any transparent material, including, but not limited to, glass or transparent plastic, or can be unglazed, bearing in mind that it is desirable that any material(s) of the window 112 and the device 100 as a whole can be readily sterilized. The window 112 can permit a user of the device 100 to visually observe the contents of buffer compartment 120. However, in other embodiments (not shown), the window 112 can show only a portion of the contents of the buffer compartment, can be omitted, or can be replaced with a mechanical, electrical, electromechanical, or electronic apparatus configured to report information about the contents of the buffer compartment 120 to a user of the device 100.

The embodiment shown in FIG. 1 also includes a dial 172 and a retaining member 180, which will be described in more detail below.

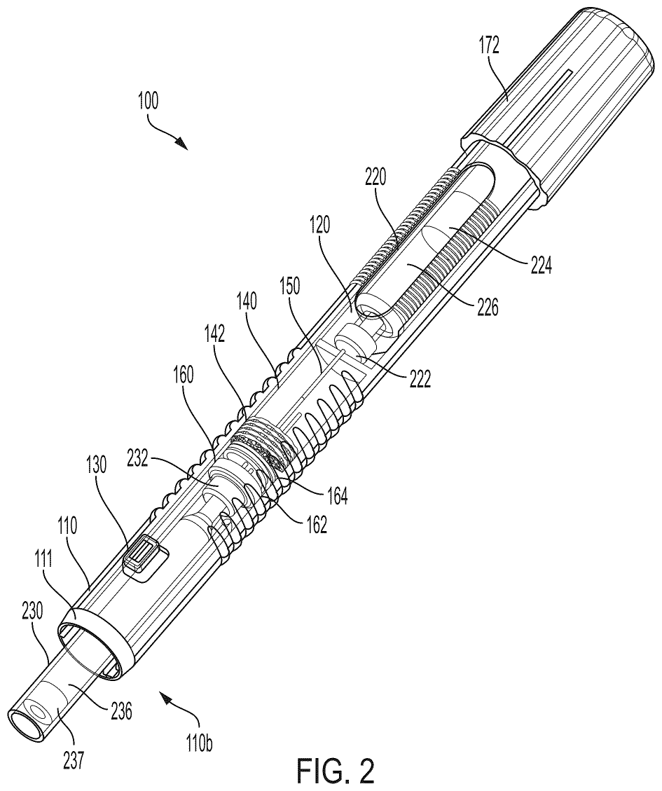

FIG. 2 shows a cutaway perspective view of the device 100. The housing 110 comprises three compartments 120, 130, and 140, namely, a buffer compartment 120, an anesthetic compartment 130, and an overflow compartment 140. In the depicted embodiment, the buffer compartment 120 is proximal to the dial 172; the anesthetic compartment 130 is distal to the dial 172; and the overflow compartment 140 is intermediate between the buffer compartment 120 and the anesthetic compartment 130. However, in other embodiments (not shown), the compartments 120, 130, and 140 can be positioned in other arrangements relative to one another.

The figures show the housing 110 as being essentially cylindrical. Further, the compartments 120, 140, and 130 are shown as consecutive and colinear from the first end 110a to the second end 110b. Although a cylinder is a convenient shape and a consecutive and colinear arrangement of compartments is a convenient arrangement, in other embodiments (not shown), the housing 110 can have any three-dimensional shape, including, but not limited to, a cylinder, a rectangular prism, a cube, a rectangular pyramid, a square pyramid, a triangular pyramid, a sphere, or another shape. Alternatively, or in addition, the arrangement of the compartments 120, 130, 140 need not be consecutive and/or colinear, but can also be selected from parallel, partially parallel, partially non-colinear, non-colinear, or have any other arrangement.

The buffer compartment 120 is configured to receive a buffer cartridge 220 comprising a first septum 222 sealing an end of the buffer cartridge 220 and a plunger 224 slidably sealing the other end of the buffer cartridge 220, and containing a buffer in the interior volume 226 of the buffer cartridge.

The buffer cartridge can have any desired size. For example, the buffer cartridge can have an interior volume 226 of 3 ml or 1.7 ml, among other volumes.

The wall of the buffer cartridge 220 can be formed from any suitable material, including, but not limited to, glass or plastic. The first septum 222 can be formed from rubber or any other suitable material. The plunger 224 can be formed from any suitable material, including, but not limited to, rubber.

Typically, the buffer is a bicarbonate buffer, although the disclosure is not so limited. In embodiments, the buffer cartridge 220 is filled with a solution of bicarbonate buffer that will evolve carbon dioxide at room temperature and pressure. Evolution of the carbon dioxide is inhibited by storing the buffer cartridge 220 in a configuration that does not allow the plunger to displace outward as carbon dioxide evolves out of solution. One method can be to apply a force to the plunger 224, where the force is sufficient to pressurize the bicarbonate buffer solution at a pressure which inhibits the evolution of carbon dioxide, thus stabilizing the pH and composition of the buffer. In exemplary embodiments, the buffer comprises sodium bicarbonate having a pH in the range from 7.5 to 7.8 and the applied pressure is above 1.2 atmospheres. In further exemplary embodiments, the force is applied by loading the buffer cartridge 220 into buffer compartment 120 with a spring, impeller, or other compression member against the plunger and maintaining the pressure until the buffer cartridge 220 is used. In an embodiment, an impeller 178 is in contact with plunger 224. Preferably, the buffer cartridge 220 is filled with all gases evacuated (the headspace eliminated) to further stabilize the pH and carbonate content of the solution.

In embodiments, the buffer is sodium bicarbonate, including, but not limited to, about 8.4% sodium bicarbonate in aqueous solution. A buffer capable of increasing the pH of the anesthetic to about 7.4 can be utilized.

In the depicted embodiment, the device 100 can be configured such that the buffer cartridge 220 is placed in the buffer compartment 120 during manufacture, with subsequent manufacturing steps rendering the buffer cartridge 220 essentially unable to be removed by a person's hands. However, in other embodiments (not shown), the device 100 can be configured such that the buffer cartridge 220 can be placed in the buffer compartment 120 after manufacture and/or can be removable by a user.

The anesthetic compartment 130 is configured to receive an anesthetic cartridge 230 comprising a second septum 232 and containing an anesthetic in the interior volume 236 of the anesthetic cartridge 230. In the depicted embodiment, the anesthetic compartment 130 is open at the second end 110b of the housing 110, and the anesthetic compartment 130 is configured to receive the anesthetic cartridge 230 by inserting the anesthetic cartridge 230 into open second end 110b. In an embodiment, the anesthetic cartridge 230 locks in place when inserted correctly. In an embodiment, correct insertion is accompanied by an audible indication, such as a click. In an embodiment, the anesthetic cartridge 230 is inserted by placing the anesthetic cartridge 230 into the device at open second end 110b and applying gentle pressure until the anesthetic cartridge 230 locks in place. In other embodiments (not shown), the device 100 can be configured such that the anesthetic cartridge 230 can be inserted into the anesthetic compartment 130 from a side of the device 100, including, but not limited to, by way of example only, through a door that can be reversibly flipped, slid, or other otherwise moved between an open and a closed conformation.

The anesthetic cartridge 230 can have many features in common with the buffer cartridge 220. For example, the anesthetic cartridge 230 can have any desired size. Also, for example, the anesthetic cartridge 230 can have an interior volume 236 of 1.8 ml or more, among other volumes. For another example, the wall of the anesthetic cartridge 230 can be formed from any suitable material, including, but not limited to, glass or plastic. The second septum 232 can be formed from rubber or any other suitable material. The anesthetic cartridge 230 can also comprise a plunger 237, which can be formed from any suitable material, including, but not limited to, rubber.

The anesthetic cartridge 230 can comprise an anesthetic formulation, typically a solution comprising from about 2% to about 4% of a desired anesthetic. In embodiments, the anesthetic formulation can be about 2% lidocaine, 1:100,000 epinephrine; about 2% lidocaine, 1:50,000 epinephrine; about 2% articaine, 1:100,000 epinephrine; about 2% articaine, 1:200,000 epinephrine; about 2% mepivacaine; about 4% prilocaine; or about 2% prilocaine, 1:200,000 epinephrine. In various embodiments, the anesthetic can be formulated as a hypoosmotic solution, such that the addition of a hyperosmotic buffer will result in a desired osmolarity, which can be closer to or equal to isosmoticity, or the osmolarity of the human body. In an embodiment, the buffer is hyperosmotic and the anesthetic is hypoosmotic, such that when the buffer and anesthetic are combined the combination has an intended target osmolarity. In an embodiment, the target osmolarity is between 200 mOsm/L and 416 mOsm/L. In an embodiment, the target osmolarity is substantially isosmotic at 308 mOsm/L.

In a further embodiment of the invention, the local anesthetic cartridge would contain an anesthetic formulated as a hypoosmotic solution (when compared to the osmolarity of human blood or tissues) such that when a hyperosmotic buffering solution, such as sodium bicarbonate, were combined with the hypoosmotic local anesthetic solution, the combined solution would be closer to isosmotic, which would allow for a greater degree of pH buffering with the buffered solution being more easily tolerated.

The overflow compartment 140 is configured to receive anesthetic expelled from the anesthetic cartridge 230 during operation of the device 100. Desirably, the overflow compartment 140 has a volume at least as large as the interior volume 226 of the buffer cartridge 220. The overflow compartment 140 will be described in more detail herein.

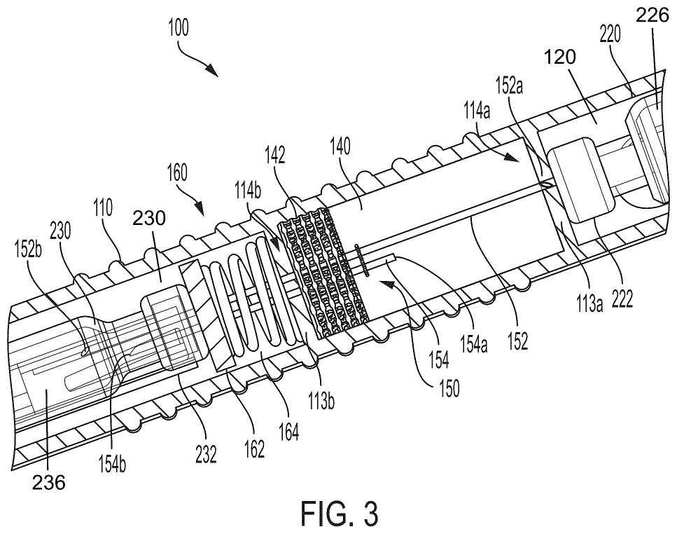

FIG. 3 shows that the buffer compartment 120, the anesthetic compartment 130, and the overflow compartment 140 are defined by the housing 110 and intercompartment walls, e.g., first intercompartment wall 113a and second intercompartment wall 113b. Both intercompartment walls 113a and 113b contain needle passages, e.g., the first intercompartment wall needle passage 114a and the second intercompartment wall needle passage 114b. Generally, the needle passages 114a and 114b have a size, shape, and position such that transfer needle 152 can reversibly pierce septum 222 to provide a fluid connection from interior volume 226 of the buffer cartridge 220 to interior volume 236 of the anesthetic cartridge 230 and exhaust needle 154 can pass through intercompartment wall 113b to provide a fluid path between the interior volume 236 of anesthetic cartridge 230 and the interior of overflow compartment 140 during use of the device 100, as will be described in more detail herein.

As can be seen in FIG. 3, there can be an assembly 150 comprising transfer needle 152 having a first end 152a and a second end 152b, the assembly 150 also comprising an exhaust needle 154, having a first end 154a and a second end 154b. In an embodiment, and as shown, the needles 152 and 154 are positioned in a parallel orientation to each other with the second end of the transfer needle 152b extending further into the interior volume 236 of anesthetic cartridge 230 than the second end of exhaust needle 154b.

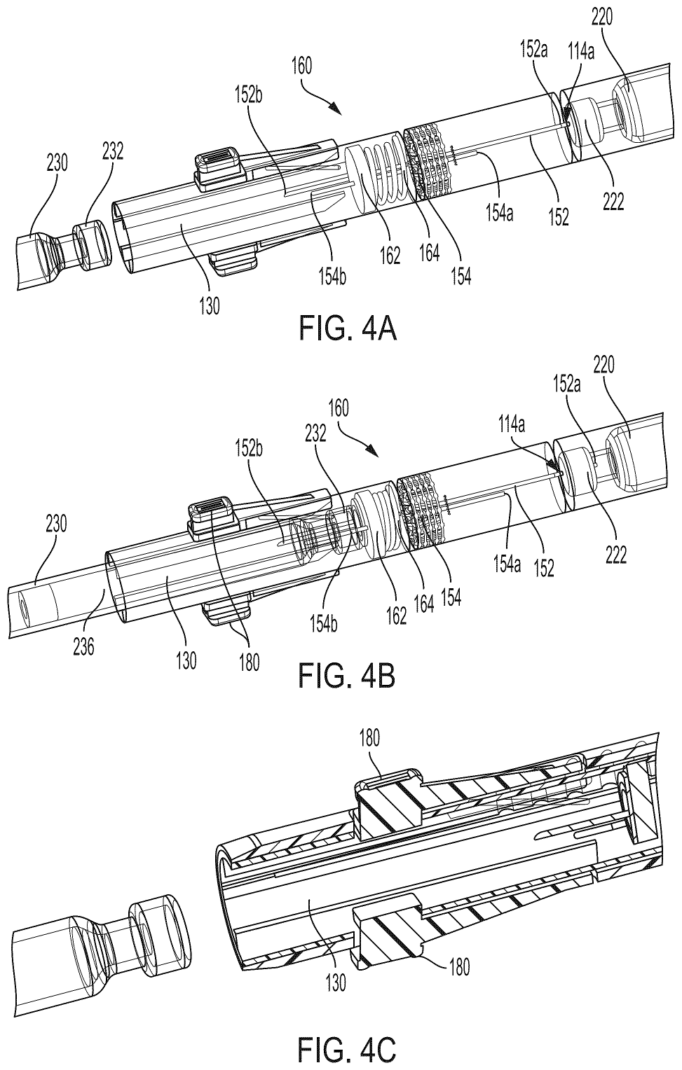

In a first conformation of the device, wherein the anesthetic chamber 130 is empty of an anesthetic cartridge 230, including, but not limited to, that shown in FIG. 4A, the second ends 152b and 154b of the transfer needle 152 and the exhaust needle 154 are positioned in the anesthetic chamber 130. Transfer needle 152 is positioned to pass through needle passage 114a, and the first end 154a of the exhaust needle 154 is positioned in the overflow compartment 140.

FIG. 2 and FIG. 3 also show a compressible member 160. As depicted in the figures, the compressible member 160 can comprise a pusher plate 162 and a spring 164. The spring 164 is positioned such that the spring 164 compresses when pressure is applied to the pusher plate 162, including, but not limited to, pressure applied by an anesthetic cartridge 230 inserted into the anesthetic compartment 130.

The compressible member 160 can be configured such that the transfer needle 152 and the exhaust needle 154 maintain a fixed position and orientation relative to the pusher plate 162 regardless of the compression state of the spring 164. As the spring 164 compresses and decompresses, the position of the transfer needle 152 and the exhaust needle 154 can move relative to other features of the device 100, including, but not limited to, the buffer compartment 120 and overflow compartment 140.

FIG. 4B shows a second conformation of the device 100, wherein the anesthetic chamber 130 contains an anesthetic cartridge 230, and the anesthetic cartridge 230 has compressed the compressible member 160. The second end 152b of the transfer needle 152 has penetrated the septum 232 and is positioned in the interior volume 236 of the anesthetic cartridge 230. The first end 152a of the transfer needle 152 has penetrated the first septum 222 of buffer cartridge 220 and is positioned in the interior volume 226 of the buffer cartridge 220. The second end 154b of exhaust needle 154 has penetrated the septum 232 and is positioned in the interior volume 236 of the anesthetic cartridge 230. The first end 154a of the exhaust needle 154 remains positioned in the overflow compartment 140. In other words, the exhaust needle 154 does not penetrate the septum 222 on the buffer cartridge 220.

Accordingly, upon compression of the compressible member 160 by the anesthetic cartridge 230, a first fluid communication channel is established between the interior volume 226 of the buffer cartridge 220, which contains buffer, and the interior volume 236 of the anesthetic cartridge 230, which contains an anesthetic solution. A second fluid communication channel is also established between the interior volume 236 of the anesthetic cartridge 230 and the overflow compartment 140.

As shown in FIG. 4B and FIG. 4C, the retaining members 180 can retain the anesthetic cartridge 230 in position such that the first and second fluid communication channels are maintained until a user disengages the retaining members 180.

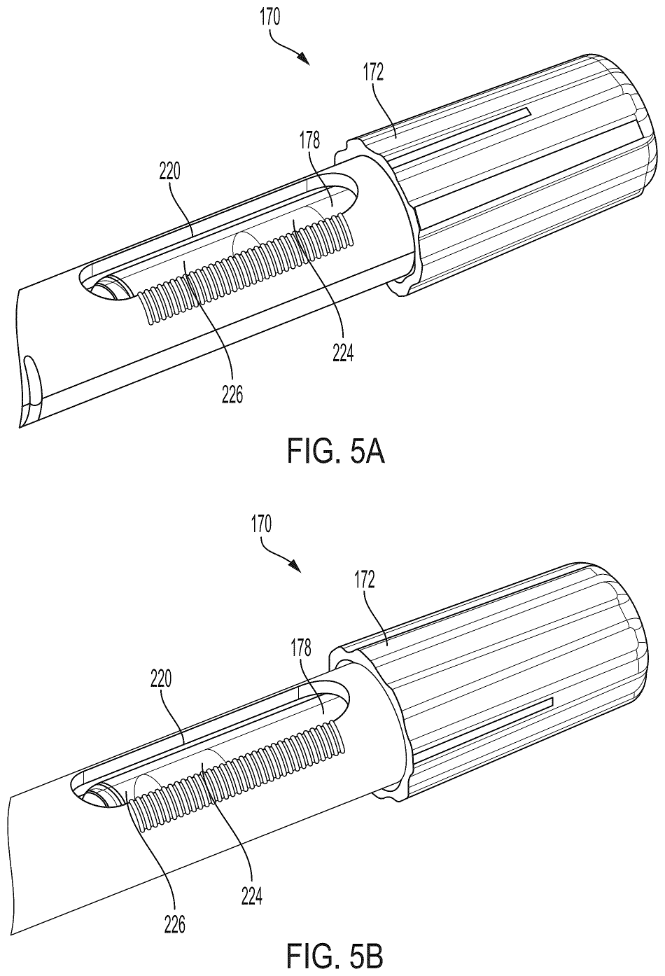

As shown in FIGS. 5A and 5B, the device 100 also can comprise an advancer member 170 configured to engage an impeller 178 with the plunger 224 upon reception of the buffer cartridge 220 by the buffer compartment 120. The configuration can be such that advancement of the impeller 178 against the plunger 224 of buffer cartridge 220 expels buffer solution from the buffer cartridge 220 into the anesthetic cartridge 230 via the transfer needle 152, whereby fluid is also expelled from the anesthetic cartridge 230 into the overflow compartment 140 via the exhaust needle 154. Simultaneously introducing buffer solution into the anesthetic cartridge while expelling the anesthetic solution into the overflow compartment allows a predetermined volume of buffer to be introduced into the anesthetic cartridge 230 without adding volume to or overfilling the anesthetic cartridge 230.

In embodiments, the advancer member 170 comprises a dial 172, wherein rotation of the dial 172 about a center axis of the housing 110 advances the impeller 178 against the plunger 224, thereby expelling buffer from the interior volume 226 of the buffer cartridge 220. FIG. 5A and FIG. 5B show such an embodiment in a first stage, FIG. 5A, before the dial 172 is rotated and a second stage, FIG. 5B, after the dial 172 has been rotated.

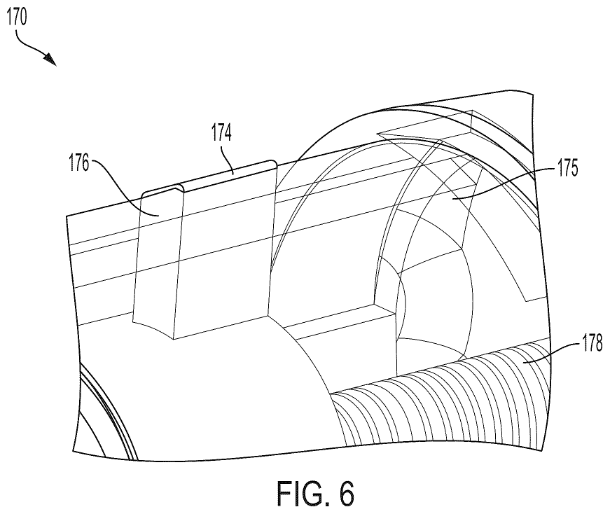

In particular embodiments, including, but not limited to, that shown in FIG. 5A, FIG. 5B, and FIG. 6, the advancer member 170 can comprise an impeller 178. The impeller 178 can be geared to the dial 172 at any desired gearing ratio, whereby turning the dial 172 by a first rotational distance advances the impeller 178 by a second, proportional axial distance. The volume of the buffer expelled from the buffer cartridge 220 can then be a function of the second distance times the cross-sectional area of the interior volume 226 of the buffer cartridge 220 in a plane perpendicular to the axis of advance of the impeller 178. Hence, the volume of the buffer expelled from the buffer cartridge 220 can be controlled by controlling the amount of rotation of the dial 172.

The amount of rotation of the dial 172 can be further controlled by a ratchet and pawl mechanism, comprising ratchet 175 and pawl 176. The dial 172 can be rotated in the direction of permitted motion of the pawl 176 relative to the ratchet 175, until the pawl 176 enters a detent notch 174. The ratchet 175 can be constructed with a single detent notch 174 (as shown in FIG. 6) or constant spacing between plural detent notches 174 (not shown). In an embodiment, the one or more detent notches can be dentate notches. Accordingly, each relative rotational advance of the pawl 176 from a first detent notch 174 to a next detent notch 174 of the ratchet 175 can be essentially constant, with the corresponding advance of the impeller 178 and corresponding amount of the buffer expelled from the buffer cartridge 220 into the anesthetic cartridge 230 also being essentially constant. Accordingly, each rotation of the dial 172 to the next detent notch 174 can mix an essentially consistent amount of buffer into the anesthetic solution in the anesthetic cartridge 230. Hence, reproducibility of buffering of anesthetic solution from one use of the device 100 to the next can be maintained.

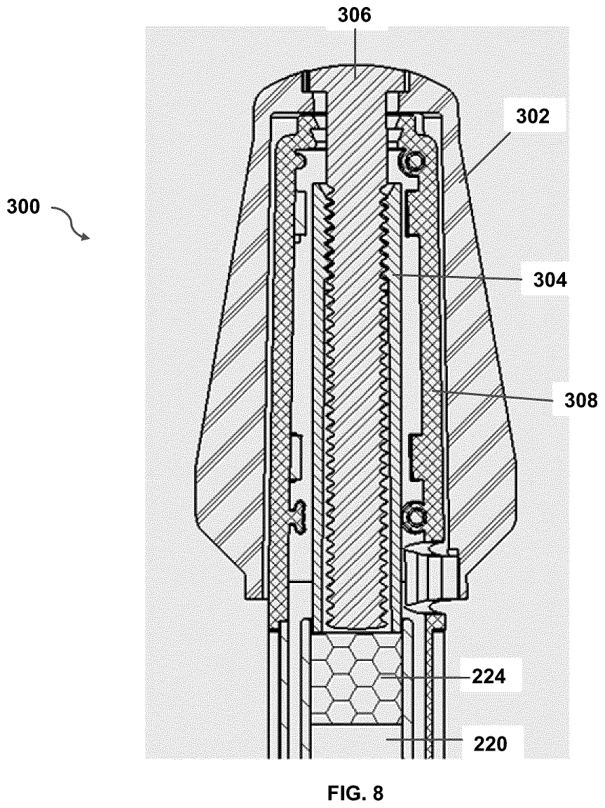

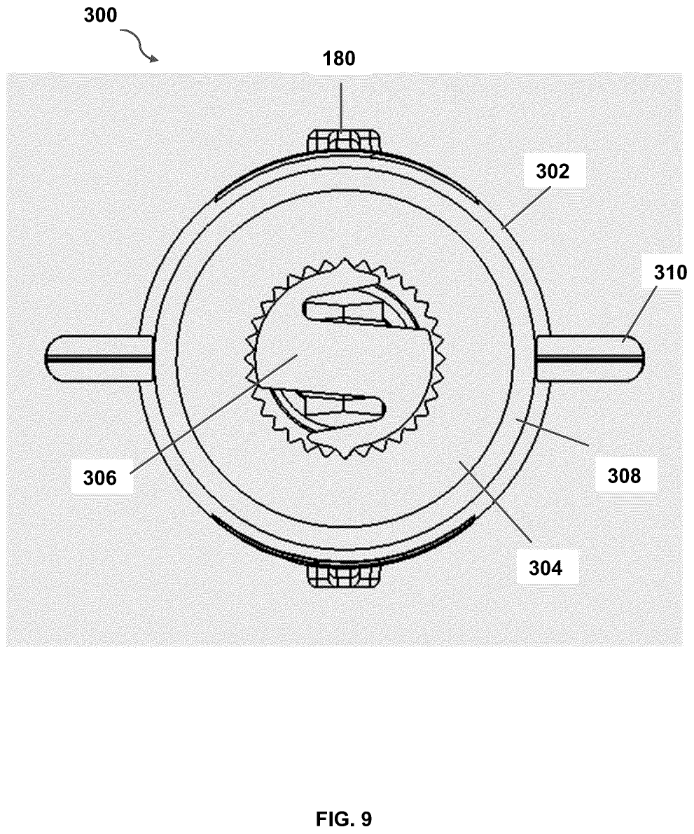

In embodiments of the device, the slip clutch mechanism 300 is designed to hold the plunger 224 in place. See FIG. 8 and FIG. 9. When an anesthetic cartridge is not present in the device to establish a fluid path, the frictional forces are overcome due to fluid incompressibility and the distal portion of the screw 306 spins while emitting an audible indicator that no engagement has taken place. See FIG. 8 and FIG. 9. In an embodiment, the slip clutch mechanism 300 prevents the screw 306 from being able to reverse and relieve pressure on the buffer solution. In the embodiment shown in FIG. 8, the slip clutch mechanism 300 is present in the interior of knob 302. The proximal portion of screw 306 acts as an impeller to advance against the plunger 224 of the buffer cartridge 220 when an anesthetic cartridge is present. During device use with an anesthetic cartridge, the distal portion of screw 306 locks with the knob 302. A portion of screw 306 interacts in a threaded manner with the casing 304 for at least a portion of the body of screw 306. Frame 308 is located interior to knob 302 and exterior to casing 304. FIG. 9 is a sectional view of the distal end of the device. Screw 306 is visible interacting with casing 304. The frame 308 is located exterior to casing 304. Knob 302 is located around the outer circumference of frame 308. Retaining members 180 and grips 310 are visible at locations on the circumference of the device.

In embodiments of the device 100, the buffer cartridge 220 can comprise sufficient buffer to buffer up to about 30 cartridges of anesthetic, including, but not limited to, one, two, three, four, five, six, seven, eight, nine, 10, 11, 12, 13, 14, 15, 16, 17, 18, 19, 20, 21, 22, 23, 24, 25, 26, 27, 28, 29, 30, or more doses of buffer. When all or essentially all of the buffer in the buffer cartridge 220 has been exhausted, the device 100 can be discarded, or the device can be discarded when only a portion of the buffer in buffer cartridge 220 has been exhausted. If a window 112 (FIG. 1) is included in the housing 110, the amount of the buffer remaining in the buffer cartridge 220 can be monitored. Thirty doses of buffer is an amount commonly sufficient to be used by a dentist over the course of a week. In other embodiments, at the time of first use, the buffer cartridge 220 can comprise more or less buffer.

When buffer is expelled from the buffer cartridge 220 into the anesthetic cartridge 230 by the action of the advancer member 170, a corresponding volume of anesthetic solution must be expelled from the anesthetic cartridge 230 into the overflow compartment 140.

The overflow compartment 140 can be empty of other features. In such embodiments, expelled anesthetic solution can have full fluid access to the entire volume of the overflow compartment 140. In doing so, it can be possible that the expelled anesthetic solution can find avenues of egress from the overflow compartment 140, e.g., the solution can traverse one or more of intercompartment wall needle passages 114a and 114b.

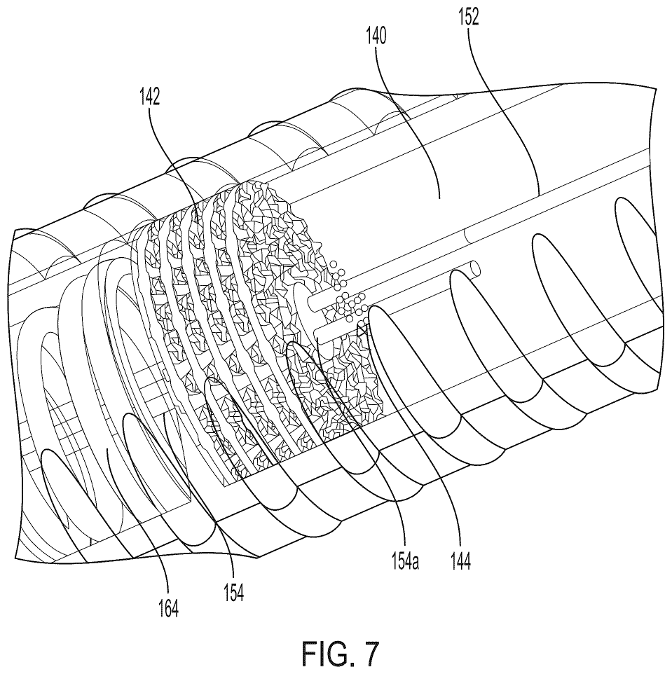

In embodiments, the device 100 can further comprise an absorbent member 142. The absorbent member 142 can be configured to absorb at least some fluid expelled from the anesthetic cartridge 230. In the depicted embodiment of e.g., FIG. 3 and FIG. 7, the absorbent member 142 is positioned in the overflow compartment 140. Alternative locations of the absorbent member 142 can be used (not shown). The absorbent member 142 can comprise any material capable of absorbing anesthetic solution. Exemplary materials include, but are not limited to, sponge, cotton, and synthetic polymers, among others.

In an embodiment, the device 100 can further comprise a plurality of granules 144 configured to absorb fluid not absorbed or not retained by the absorbent member 142. As depicted in FIG. 7, the granules 144 can be positioned in the overflow compartment 140, although alternative locations (not shown) can be used. In an embodiment, the granules are of a large enough diameter to not enter the end of exhaust needle 154a. In some embodiments, the granules 144 can comprise any polymer capable of absorbing the anesthetic solution. Exemplary materials include, but are not limited to, polyacrylates, ethylene maleic anhydride, cross-linked carboxymethylcellulose, polyvinyl alcohol copolymers, and cross-linked polyethylene oxide.

Alternative embodiments can include only the granules 144 in the overflow compartment 140 without any absorbent member 142 in the overflow compartment 140. In an embodiment, the granules are of a large enough diameter to not enter the end of exhaust needle 154a.

The present disclosure also relates to methods of manufacturing a device, including, but not limited to, the device 100 described above. For example, herein is disclosed a method, comprising providing a device comprising a buffer compartment configured to receive a buffer cartridge comprising a first septum and a plunger, and containing a buffer; an anesthetic compartment configured to receive an anesthetic cartridge comprising a second septum and containing an anesthetic; an overflow compartment; a needle assembly, comprising a transfer needle having a first end and a second end and an exhaust needle having a first end and a second end; a compressible member configured to compress upon reception of the anesthetic cartridge by the anesthetic compartment, wherein compression of the compressible member penetrates the first end of the transfer needle through the first septum, the second end of the transfer needle through the second septum, the second end of the exhaust needle through the second septum, and wherein the first end of the exhaust needle is present in the overflow compartment, and an advancer member configured to engage with the plunger upon reception of the buffer cartridge by the buffer compartment, wherein advancement of the advancer member expels buffer from the buffer cartridge into the anesthetic cartridge via the transfer needle, whereby fluid is expelled from the anesthetic cartridge into the overflow compartment via the exhaust needle; and inserting the buffer cartridge into the buffer compartment. In some embodiments, the first end of the exhaust needle enters the overflow compartment. In some embodiments, the first end of the exhaust needle is already present in the overflow compartment.

Although the recitation of inserting the buffer cartridge into the buffer compartment follows the recitation of providing a device comprising the buffer compartment and other elements, the inserting is not limited to being performed after the device is otherwise fully assembled. For example, the inserting can be performed when the device is in an incomplete state of manufacture but the location and position of the buffer compartment have been established and subsequent manufacturing steps will not be impeded by the buffer cartridge.

For a further example, the housing can be a cylindrical housing formed of two halves divided through the long axis of the housing, e.g., into two semicylinders. A first half of the housing can sufficiently define the buffer compartment for the buffer cartridge to be inserted therein. The insertion of the buffer cartridge can be performed before, after, or simultaneously with the placement of various other elements in the buffer compartment, anesthetic compartment, and/or overflow compartment, e.g. before, after, or simultaneously with the needle assembly, absorber member, granules, compressible member, pressure plate, spring, advancer member, pawl, ratchet, slip clutch, impeller, and/or retaining member.

After all elements that can be placed in the first half of the housing have been placed, the second half of the housing can be placed and joined to the first half, including, but not limited to, by a snap-fit, an adhesive, or one or more screws, among other joining apparatus, or combinations thereof. A reinforcement band can be added to further secure the first half and the second half of the housing. A dial can be positioned over an end of the housing and in engagement with one or more of the advancer member, ratchet, slip clutch, and/or impeller.

After the buffer cartridge has been inserted and the device is assembled, the device can be sterilized and/or packaged. For example, the device can be packaged in combination with instructions for performing a method, the method comprising providing an anesthetic cartridge comprising a second septum and containing an anesthetic; compressing the compressible member with the anesthetic cartridge, thereby penetrating the first end of the transfer needle through the first septum, the second end of the transfer needle through the second septum, the second end of the exhaust needle through the second septum, and wherein the first end of the exhaust needle is present in the overflow compartment, and advancing the plunger, thereby expelling buffer from the buffer cartridge into the anesthetic cartridge via the transfer needle, whereby fluid is expelled from the anesthetic cartridge into the overflow compartment via the exhaust needle. In some embodiments, the first end of the exhaust needle enters the overflow compartment. In some embodiments, the first end of the exhaust needle is already present in the overflow compartment. In other words, the device can be packaged with instructions for a method of its use.

In embodiments, the instructions can further comprise instructions for withdrawing the anesthetic cartridge from the transfer needle and the exhaust needle.

In embodiments, the instructions can further comprise instructions for providing a second or additional instances of the anesthetic cartridge; and repeating the compressing and advancing with the second or additional instance of the anesthetic cartridge.

The present disclosure also relates to methods of using a device, including, but not limited to, device 100 described above. For example, herein is disclosed a method, comprising providing a buffer cartridge comprising a first septum, a plunger, and containing a buffer; providing an anesthetic cartridge comprising a second septum and containing an anesthetic; providing a needle assembly, comprising a transfer needle having a first end and a second end and an exhaust needle having a first end and a second end; compressing a compressible member with the anesthetic cartridge, thereby penetrating the first end of the transfer needle through the first septum, the second end of the transfer needle through the second septum, the second end of the exhaust needle through the second septum, and wherein the first end of the exhaust needle is present in the overflow compartment, and advancing the plunger, thereby expelling buffer from the buffer cartridge into the anesthetic cartridge via the transfer needle, whereby fluid is expelled from the anesthetic cartridge into the overflow compartment via the exhaust needle. In some embodiments, the first end of the exhaust needle enters the overflow compartment. In some embodiments, the first end of the exhaust needle is already present in the overflow compartment.

In embodiments, the method can further comprise one or more additional actions and/or a step of the method can be characterized by one or more additional features, including, but not limited to, one, two, three, four, five, six, seven, eight, nine or more of the following. In the method, advancing the plunger can comprise rotating a dial or knob.

In the method, rotating the dial or knob can engage a ratchet and pawl mechanism. In embodiments, expelling the buffer comprises expelling a predetermined dose of the buffer upon engagement of the ratchet and pawl mechanism.

In the method, a slip clutch mechanism present interior to the dial or knob prevents the screw from being able to reverse and relieve pressure on the buffer solution. In the embodiment, the slip clutch mechanism is present in the interior of the dial or knob.

The method can further comprise absorbing at least some fluid expelled into the overflow compartment by an absorber member or other absorbent material.

The method can further comprise absorbing fluid not absorbed or not retained by the absorbent member, by a plurality of granules.

The method can further comprise retaining the anesthetic cartridge in a position to maintain the compressing of the compressible member.

The method can further comprise withdrawing the anesthetic cartridge from the transfer needle and the exhaust needle.

The method can further comprise providing a second instance of the anesthetic cartridge; and repeating the compressing and advancing with the second instance of the anesthetic cartridge.

In the method, the buffer cartridge, the needle assembly, the compressible member, and the advancer member configured for advancing the plunger, can be positioned within a housing, and the method can further comprise disposing of the housing when the buffer cartridge is substantially empty of buffer.

The devices and methods disclosed herein provide one or more desirable results for dentists and personnel supporting them. The devices are generally simpler to use than previous devices, thereby reducing time spent by dentists in training and reducing the burden on customer support personnel. The devices can be preloaded with buffer, thereby reducing the number of discrete items a dentist or dental office staff member must purchase and track. The devices allow very rapid buffering of anesthetics by the method of use described herein, using buffer maintained under conditions that generally maintain its pH throughout the intended use-life of the unit. In an embodiment, the devices are disposable and are convenient for dentists to decommission at the end of the use-life, with no requirement for assembly, disassembly, or cleaning. The device has generally simpler and more unified mechanisms than prior devices, thereby improving reliability.

Although the description has described particular embodiments relating to dentistry, as will be apparent to a person of ordinary skill in the art, the device and the methods disclosed herein can be used for the buffering of any anesthetic solution for any medical use or other use where precise repeatable pH buffering is desired.

While the above is a description of embodiments, various alternatives, modifications, additions, and substitutions are possible without departing from the scope thereof, which is defined by the claims.

What is claimed is:

1. A device, comprising:
a buffer compartment configured to receive a buffer cartridge comprising a first septum and a plunger, and containing a buffer;
an anesthetic compartment configured to receive an anesthetic cartridge comprising a second septum and containing an anesthetic;
an overflow compartment;
a needle assembly, comprising a transfer needle having a first end and a second end and an exhaust needle having a first end and a second end;
a compressible member configured to compress upon reception of the anesthetic cartridge by the anesthetic compartment, wherein compression of the compressible member causes the first end of the transfer needle to penetrate the first septum, the second end of the transfer needle to penetrate the second septum, the second end of the exhaust needle to penetrate the second septum, and wherein first end of the exhaust needle is present in the overflow compartment; and
an advancer member configured to engage with the plunger upon reception of the anesthetic cartridge by the anesthetic compartment, wherein advancement of the advancer member expels buffer from the buffer cartridge into the anesthetic cartridge via the transfer needle, whereby fluid is expelled from the anesthetic cartridge into the overflow compartment via the exhaust needle.

2. The device of claim 1, wherein the advancer member comprises a dial, wherein rotation of the dial advances the plunger.

3. The device of claim 2, wherein the advancer member comprises a ratchet and pawl mechanism configured to ensure the dial rotates in only one direction and expels a predetermined dose of the buffer upon a rotation or a partial rotation of the dial.

4. The device of claim 2, wherein the advancer member comprises one or more detent notches.

5. The device of claim 2, wherein the ratchet and pawl mechanism are situated to cause an audible indication when the dial has been rotated to completion.

6. The device of claim 1, further comprising a slip clutch mechanism that locks a screw with a knob of the advancer member when the anesthetic cartridge is present in the device.

7. The device of claim 1, wherein the overflow compartment comprises an absorbent member configured to absorb at least some fluid expelled from the anesthetic cartridge.

8. The device of claim 1, wherein the overflow compartment comprises a plurality of granules configured to absorb fluid.

9. The device of claim 1, wherein the anesthetic compartment comprises at least one retaining member configured to retain the anesthetic cartridge after reception such that the compressible member remains compressed until the anesthetic cartridge is removed from the anesthetic compartment.

10. The device of claim 1, wherein the buffer compartment is proximal toa first end of the device, the anesthetic compartment is proximal toa second end of the device, and the overflow compartment is positioned between the buffer compartment and the anesthetic compartment.

11. The device of claim 1, wherein the device is cylindrical.

12. A method, comprising:
providing a device;
providing a buffer cartridge comprising a first septum, a plunger, and containing a buffer;
providing an anesthetic cartridge comprising a second septum and containing an anesthetic;
providing a needle assembly, comprising a transfer needle having a first end and a second end and an exhaust needle having a first end and a second end;
attaching the anesthetic cartridge to the device;
compressing a compressible member with the anesthetic cartridge, thereby causing the first end of the transfer needle to penetrate the first septum, the second end of the transfer needle to penetrate the second septum, the second end of the exhaust needle to penetrate the second septum, and wherein the first end of the exhaust needle is present in the overflow compartment; and
advancing the plunger, thereby expelling buffer from the buffer cartridge into the anesthetic cartridge via the transfer needle, whereby fluid is expelled from the anesthetic cartridge into the overflow compartment via the exhaust needle.

13. The method of claim 12, wherein an audible indication occurs when the anesthetic cartridge is correctly inserted in the device.

14. The method of claim 12, wherein advancing the plunger comprises rotating a dial.

15. The method of claim 14, wherein rotating the dial engages a ratchet and pawl mechanism, and wherein expelling the buffer comprises expelling a predetermined dose of the buffer upon engagement of the ratchet and pawl mechanism.

16. The method of claim 12, further comprising providing a slip clutch mechanism that locks a screw with a knob of an advancer member when the anesthetic cartridge is present.

17. The method of claim 12, further comprising:
absorbing at least some fluid expelled into the overflow compartment by a absorber member.

18. The method of claim 12, further comprising:
absorbing fluid, such as fluid not absorbed or not retained by the absorbent member if the absorbent member is present, by a plurality of granules.

19. The method of claim 12, further comprising:
retaining the anesthetic cartridge in a position to maintain the compressing of the compressible member.

20. The method of claim 12, further comprising:
withdrawing the anesthetic cartridge from the device.

21. The method of claim 12, wherein the method comprises compounding a medication.

22. The method of claim 12, wherein the buffer is hyperosmotic and the anesthetic is hypoosmotic, such that when the buffer and anesthetic are combined the combination has an intended target osmolarity.

23. The method of claim 22 wherein the target osmolarity is between 200 mOsm/L and 416 mOsm/L.

24. The method of claim 23 where the target osmolarity is substantially isosmotic at 308 mOsm/L.

25. The method of claim 12, wherein the buffer cartridge, the needle assembly, the compressible member and an advancer member configured for advancing the plunger are positioned within a housing, and the method further comprises:
disposing of the housing when the buffer cartridge is substantially empty of buffer.

26. A method, comprising:
providing a device comprising:
a buffer compartment configured to receive a buffer cartridge comprising a first septum and a plunger, and containing a buffer;
an anesthetic compartment configured to receive an anesthetic cartridge comprising a second septum and containing an anesthetic;
an overflow compartment;
a needle assembly, comprising a transfer needle having a first end and a second end and an exhaust needle having a first end and a second end;
a compressible member configured to compress upon reception of the anesthetic cartridge by the anesthetic compartment, wherein compression of the compressible member causes the first end of the transfer needle to penetrate the first septum, the second end of the transfer needle to penetrate the second septum, the second end of the exhaust needle to penetrate the second septum, and the first end of the exhaust needle is present in the overflow compartment, and an advancer member configured to engage with the plunger upon reception of the anesthetic cartridge by the anesthetic compartment, wherein advancement of the advancer member expels buffer from the buffer cartridge into the anesthetic cartridge via the transfer needle, whereby fluid is expelled from the anesthetic cartridge into the overflow compartment via the exhaust needle; and
inserting the buffer cartridge into the buffer compartment; and
packaging the device in combination with instructions for performing a method, the method comprising providing an anesthetic cartridge comprising a second septum and containing an anesthetic; compressing the compressible member with the anesthetic cartridge, thereby causing the first end of the transfer needle to penetrate the first septum, the second end of the transfer needle to penetrate the second septum, the second end of the exhaust needle to penetrate the second septum, and wherein the first end of the exhaust needle is present in the overflow compartment, and advancing the plunger, thereby expelling buffer from the buffer cartridge into the anesthetic cartridge via the transfer needle, whereby fluid is expelled from the anesthetic cartridge into the overflow compartment via the exhaust needle.
